(19) United States
(12) Patent Application Publication
(10) Pub. No.: US 2004/0122104 A1
(43) Pub. Date: Jun. 24, 2004

(54) Modified release compositions of milnacipran

(75) Inventors: Jane Hirsh, Wellesley, MA (US); Roman V. Rariy, Allston, MA (US); Shubha Chungi, Sharon, MA (US); Michael Heffernan, Hingham, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Collegium Pharmaceutical, Inc.

(21) Appl. No.: 10/691,936

(22) Filed: Oct. 23, 2003

Related U.S. Application Data
(60) Provisional application No. 60/421,640, filed on Oct. 25, 2002.
(60) Provisional application No. 60/431,626, filed on Dec. 5, 2002.
(60) Provisional application No. 60/431,627, filed on Dec. 5, 2002.
(60) Provisional application No. 60/431,906, filed on Dec. 9, 2002.
(60) Provisional application No. 60/431,861, filed on Dec. 9, 2002.
(60) Provisional application No. 60/443,618, filed on Jan. 29, 2003.
(60) Provisional application No. 60/459,061, filed on Mar. 28, 2003.
(60) Provisional application No. 60/458,994, filed on Mar. 28, 2003.
(60) Provisional application No. 60/458,995, filed on Mar. 28, 2003.

(51) Int. Cl. A61K 9/22; A61K 31/165

(52) U.S. Cl. 514/620; 424/468

(57) ABSTRACT

A once-a-day oral milnacipran modified release formulation has been developed. The formulation comprises an extended release dosage unit (optionally containing the immediate release portion) coated with delayed release coating. The milnacipran composition, when administered orally, first passes through the stomach releasing from zero to less than 10% of the total milnacipran dose and then enters the intestines where drug is released slowly over an extended period of time. The release profile is characterized by a 0.05-4 hours lag time period during which less than 10% of the total milnacipran dose is released followed by a slow or extended release of the remaining drug over a defined period of time. The composition provides in vivo drug plasma levels characterized by Tₘₐₓ at 4-10 hours and an approximately linear drop-off thereafter and Cₘₐₓ below 3000 ng/ml, preferably below 2000 ng/ml, and most preferably below 1000 ng/ml. The composition allows milnacipran to be delivered over approximately 24 hours, when administered to a patient in need, resulting in diminished incidence or decreased intensity of common milnacipran side effects such as sleep disturbance, nausea, vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

This application claims priority under 35 U.S.C. 119 to U.S. Ser. No. 60/421,640 filed Oct. 25, 2002; U.S. Ser. No. 60/431,626 filed Dec. 05, 2002; U.S. Ser. No. 60/431,627 filed Dec. 05, 2002; U.S. Ser. No. 60/431,906 filed Dec. 09, 2002; U.S. Ser. No. 60/431,861 filed Dec. 09, 2002; U.S. Ser. No. 60/443,618 filed Jan. 29, 2003; U.S. Ser. No. 60/459,061 filed Mar. 28, 2003; U.S. Ser. No. 60/458,994 filed Mar. 28 2003; and U.S. Ser. No. 60/458,995 filed Mar. 28, 2003.

### FIELD OF THE INVENTION

The present invention generally relates to milnacipran modified release compositions.

BACKGROUND OF THE INVENTION

Efficacy and tolerability are important factors determining the choice of a medication for treatment of mental depression and other mental disorders including Functional Somatic Disorders. The move from tricyclic antidepressants (TCAs) to selective serotonin reuptake inhibitors (SSRIs) involved not only the loss of the direct receptor interactions responsible for the adverse side effects of TCAs, but also the ability to inhibit the reuptake of norepinephrine. Selectivity for the single neurotransmitter, serotonin, may explain why SSRIs tend to be less efficacious than the TCAs, especially in more serious forms of depression (Lopez-Ibor J. et al., 1996, Int. Clin. Psychopharm., 11:41-46). Older TCAs are associated with significant behavioral toxicity, notably psychomotor and cognitive impairment and sedation. SSRIs are largely devoid of these effects, but gastrointestinal disturbances such as nausea and dyspepsia are common with these agents (Hindmarch I., 1997, Human Psychopharmacology, 12:115-119). For example, for widely prescribed SSRI sertraline (Zoloft®, Pfizer) the top three adverse events associated with discontinuation of treatment were nausea, insomnia, and diarrhea (Physician's Desk Reference, 57^{th} Edition, 2003, Thomson Medical).

Efforts toward improving antidepressant medications are guided by cumulative evidence from neurochemical and clinical studies supporting the therapeutic potential of enhancing monoamine function in depression. A number of antidepressant drugs, serotonin and norepinephrine reuptake inhibitors (SNRIs), including duloxetine, venlafaxine, and milnacipran, have been developed based on their interaction with both serotonin (5-HT) and norepinephrine (NE) receptors. Milnacipran is more appropriately referred to as norepinephrine and serotonin reuptake inhibitor (NSRI) since its norepinephrine (“NE”) to serotonin (“5-HT”) ratio is 2:1 (Moret et al., 1985, Neuropharmacology, 24:1211-1219; Palmier et al., 1989, Eur. J. Clin. Pharmacol., 37:235-238). Current clinical evidence suggests that these new agents may offer improved efficacy and/or faster onset of action compared with SSRIs (Tran P. V. et al., 2003, J. Clin. Psychopharmacol., 23:78-86). Recent trials with NSRI milnacipran suggest that this compound is effective in relieving pain both associated with, and independent of, depression (Briley M., 2003, Curr. Opin. Investig. Drugs, 4:42-45; Cypress Bioscience Inc., Cypress Bioscience Inc. Announces Final Results of Milnacipran Phase II Clinical Trial in Fibromyalgia, Media Release, Mar. 21, 2003, Available from: URL: http://www.cypressbio.com).

Unfortunately these SNRI and NSRI compounds have demonstrated numerous side effects in human clinical trials.

For example, the safety and tolerability of duloxetine (Cymbalta®, Eli Lilly and Company) was assessed in a pooled analysis of 7 double-blind trials involving 1032 patients treated with duloxetine (40-120 mg/day) and 732 patients treated with placebo. Adverse events which occurred at a rate of more than 5% for duloxetine were nausea, dry mouth, fatigue, dizziness, constipation, somnolence, decreased appetite, and sweating. Adverse events which led to discontinuation of treatment were nausea, dizziness, somnolence, dermatitis, insomnia, headache, and fatigue. Nausea and dizziness led to significantly more duloxetine-treated patients discontinuing treatment, compared with placebo (Mallinckrodt C. et al., American Psychiatric Association 2002 Annual Meeting, New Research Abstracts, 119, May 18, 2002; Detke M. J. et al., American Psychiatric Association 2002 Annual Meeting, New Research Abstracts, 33-34, May 18, 2002). Nausea was the only adverse event reported as a reason for discontinuation (Eli Lilly and Company, New Research Shows Cymbalta Reduces Anxiety Symptoms Associated With Depression, Media Release: Sep. 18, 2003).

For venlafaxine (Effexor®, Wyeth-Ayerst), a member of the SNRI family, major reported side effects are the ones that affected the gastrointestinal system. In 4- to 8-week placebo-controlled clinical trials treatment-emergent major gastrointestinal adverse experience incidence for Effexor® versus placebo (n=1,033 vs. 609) were: nausea (37% vs. 11%), constipation (15% vs. 7%), anorexia (11% vs. 2%), and vomiting (6% vs. 2%). In the same clinical trials treatment-emergent major central nervous system adverse experience incidence were: somnolence (23% vs. 9%), dry mouth (22% vs. 11%), dizziness (19% vs 7%), insomnia (18% vs. 10%), nervousness (13% vs. 6%), anxiety (6% vs. 3%), tremor (5% vs. 1%). Importantly, nausea, in addition to being the most common reported side effect (see above), was the top reason venlafaxine patients in Phase 2 and Phase 3 depression studies discontinued treatment: almost 32% of patients who discontinued treatment did so due to nausea (Physician's Desk Reference, 57th Edition, 2003, Thomson Medical).

Milnacipran (Ixel®, Pierre Fabre), has demonstrated numerous adverse reactions in human clinical trials with tolerability decreasing with increasing dose (Puech A. et al., 1997, Int. Clin. Psychopharm., 12:99-108). In the double-blind, randomized, multicenter clinical study the most frequent spontaneously reported adverse events for 100 mg/day milnacipran twice daily were as follows: abdominal pain (13%), constipation (10%), and headache (9%). Interestingly, when in the same study milnacipran was given 200 mg/day twice daily, pain related adverse reactions decreased (headache to 8% and abdominal pain to 7%) but nausea and vomiting were more pronounced side effects and were reported by 7% of the patients (Guelfi J. D., 1998, Int. Clin. Psychopharm., 13:121-128). In a double-blind comparative study involving 219 elderly patients with depression the only adverse event reported more frequently for milnacipran recipients than for TCA imipramine recipients was nausea. Patients received either milnacipran or imipramine 75-100 mg/day twice daily for 8 weeks (Tignol J. et al., 1998, Acta Psychiatrica Scandinavica, 97:157-165). It was also observed that when milnacipran was administered intravenously to 10 patients, five of them reported transient nausea. Nausea was primarily reported at the moment of peak of milnacipran plasma level (Caron J. et al., 1993, Eur. Neuropsychopharmacol., 3:493-500). This study clearly demonstrates that nausea is directly correlated with the milnacipran blood plasma concentration. In addition, it strongly suggests that the nausea can be a centrally mediated side effect since the drug was given intravenously in this study. Data from other studies suggest that milnacipran may also induce a locally mediated nausea via gastric irritation (the rapid onset of the nausea was observed even prior to achieving peak plasma levels).

The incidence of spontaneously reported milnacipran adverse experiences in placebo-controlled clinical trials is given in Table 1 (adverse effect is listed if frequency was more than 2% in milnacipran 100 mg/day group). As it can be clearly seen from data presented in Table 1, the incidence of certain adverse events increases with dosage, including nausea, vomiting, sweating, hot flashes, palpitations, tremor, anxiety, dysuria, and insomnia.

TABLE 1
Incidence of spontaneously reported milnacipran adverse
experiences in placebo-controlled clinical trials

| | Frequency of Adverse Experiences (%) | | | |
|---|---|---|---|---|
| | | 50 mg/day | 100 mg/day | 200 mg/day |
| Adverse | Placebo | twice daily | twice daily | twice daily |
| Event | N = 394 | N = 426 | N = 1871 | N = 865 |
| Nausea | 10.9 | 12.7 | 11.2 | 19.4* |
| Headache | 17.0 | 14.6 | 8.4 | 13.5 |
| Increased | 1.3 | 14.0 | 4.3* | 11.6* |
| Sweating | | | | |
| Constipation | 4.3 | 8.0 | 6.5 | 11.4* |
| Insomnia | 10.7 | 9.2 | 6.1 | 11.3 |
| Dry mouth | 5.6 | 9.4 | 7.9 | 9.0 |
| Vomiting | 3.6 | 3.8 | 3.9 | 7.9* |
| Abdominal | 5.1 | 6.1 | 6.5 | 7.6 |
| Pain | | | | |
| Tremor | 1.5 | 0.9 | 2.5 | 6.7* |
| Anxiety | 1.3 | 2.8 | 4.1 | 5.1 |
| Palpitations | 1.8 | 2.3 | 2.7 | 4.6 |
| Vertigo | 1.8 | 1.6 | 5.0 | 4.5 |
| Fatigue | 3.0 | 2.8 | 2.5 | 4.4 |
| Dysuria | 0.3 | 1.4 | 2.1* | 3.7* |
| Hot flushes | 0 | 1.6 | 3.0 | 3.6 |
| Somnolence | 3.8 | 5.4 | 2.3 | 3.5 |
| Agitation | 3.0 | 1.6 | 3.3 | 2.9 |
| Nervousness | 2.0 | 4.2 | 2.0 | 2.8 |
| Dyspepsia | 4.1 | 3.5 | 2.1 | 2.2 |

*Significantly greater than placebo

It is important to note that in one of the early depression trials, even after one week of milnacipran dose escalation employed to reduce side effects, the most commonly reported reason for discontinuation of treatment because of adverse effects was nausea and vomiting (Leinonen E., 1997, Acta Psychiatr. Scand., 96:497-504). In the recent fibromyalgia clinical trial with the long dose escalation period (four weeks) which was implemented in order to reduce milnacipran side effects and increase patient's tolerance, the most common dose-related side effect reported by patients was nausea (Cypress Bioscience Inc., Cypress Bioscience Inc. Announces Final Results of Milnacipran Phase II Clinical Trial in Fibromyalgia, Media Release, Mar. 21, 2003).

The data presented in Table I demonstrates that the currently available immediate release formulation of milnacipran is not ideal for the treatment of health conditions that require milnacipran doses equal or above 100 mg/day given either as once a day or twice a day due to high incidence of treatment-emergent side effects that leads to poor patient tolerance. Higher doses are required in the treatment of severe depression and other associated disorders. As shown in one of the early antidepressant clinical trials, milnacipran dosage of 200 mg/day was superior to the lower doses (Von Frenckell R et al., 1990, Int. Clin. Psychopharmacology 5:49-56). Milnacipran dosing regime of 100-250 mg daily was recently reported for the treatment of fibromyalgia (U.S. Pat. No. 6,602,911). It would be very difficult to reach the upper limits of the dose range using the currently available formulation due to the dose related treatment emergent side effects and the need to titrate over a long period to reach the required dose.

Moreover, an immediate release formulation of milnacipran may not be suitable for a once-daily dosing regimen for a treatment of depression due to milnacipran's relatively short, approximately 8 hours, half-life (Ansseau M. et al., 1994, Psychopharmacology 114:131-137). Milnacipran's half-life could also be responsible for the fact that twice-a-day administration (versus once-a-day) of immediate release formulation in fibromyalgia trial resulted in pain improvement statistically superior to that of placebo treatment (Cypress Bioscience Inc., Cypress Bioscience Inc. Announces Final Results of Milnacipran Phase II Clinical Trial in Fibromyalgia; Media Release, Mar. 21, 2003).

Merely stating that a drug can be administered using a sustained release formulation is not sufficient. For example, U.S. Pat. No. 6,602,911 to Kranzler, et al. states “for administration orally, the compounds may be formulated as a sustained release preparation”. While the above patent references formulation techniques, only WO98/08495 by Paillard B. et al. provides specific sustained release formulations of milnacipran. Moreover, no reference is made by Paillard regarding diminishing locally and/or centrally mediated side effects. Only by careful understanding of the relationship of the therapeutic dose to plasma levels can a modified dosage form be designed that will reduce, diminish, or prevent locally mediated as well a centrally mediated side effects. WO 98/08495 refers to a prolonged release formulation of milnacipran dosage ranging from 60-240 mg and releasing 10-55% of the total dose within two hours, consisting of saccharose and/or starch minigranules coated with the active drug and then coated with at least one polymer insoluble in water but permeable in physiological fluids.

U.S. Pat. No. 6,066,643 by Perry K., provides a method of potentiating the therapeutic action of an SSRI where milnacipran is administered with monoxidine. Perry suggests alleviating or diminishing side effects of a SSRI by co-formulating SSRI in a “quick, sustained, or delayed release” formulation with a centrally acting antihypertensive agent. The administration of the latter compound to humans is associated with drowsiness, headache and dry mouth. Perry's approach may result in additional side effects experienced by patients.

It is therefore an object of the present invention to provide milnacipran formulations which will lower incidence and intensity of side effects, especially for higher dosages, and lower or reduce the frequency of dosing and the need to slowly titrate the drug in order to get to the therapeutic dose levels required for treatment of these disorders.

It is therefore an object of the present invention to provide milnacipran formulations that produce a therapeutic effect over approximately 24 hours when administered to a patient in need, wherein the release rate and dosage are effective to provide relief from at least one disorder selected from the group consisting of depression, fibromyalgia syndrome, chronic fatigue syndrome, pain, attention deficit/hyperactivity disorder, and visceral pain syndromes (VPS), such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache, with diminished incidence and reduced intensity of common milnacipran side effects reported for immediate release formulation.

It is a further object of the present invention to provide formulations that provide alternative pharmacokinetic release profiles that eliminate or diminish unwanted side effects and the current need to slowly increase (titrate) doses in order to achieve the desired therapeutic dose.

It is still another object of the present invention to provide a formulation that provides a unit dose between 25 and 500 mg which provides for flexibility in morning or evening administration.

### SUMMARY OF THE INVENTION

A once-a-day oral milnacipran modified release composition has been developed. The milnacipran composition, when administered orally, first passes through the stomach releasing from zero to less than 10% of the total milnacipran dose and then enters the intestines where drug is released slowly over an extended period of time. The release profile is characterized by a 0.05 to four hour lag time period during which less than 10% of the total milnacipran dose is released into the stomach followed by a slow or extended release within the intestines of the remaining drug over a defined period of time. The composition provides in vivo drug plasma levels characterized by Tₘₐₓ at 4-10 hours and, optionally, an approximately linear drop-off thereafter, and Cₘₐₓ below 3000 ng/ml, preferably below 2000 ng/ml, and most preferably below 1000 ng/ml. These levels help to avoid stimulation of the cholinergic effects on the CNS. The composition delivers milnacipran over approximately 24 hours, resulting in diminished incidence and decreased intensity of common milnacipran side effects such as nausea, vomiting, sleep disturbance, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

### BRIEF DESCRIPTION OF THE DRAWINGS

FIG. 1 is a graph of the mean milnacipran blood plasma concentration (PPB) over time (hours) following administration of delayed release/extended release milnacipran formulation (120 mg milnacipran hydrochloride per tablet) to male healthy human subjects.

### DETAILED DESCRIPTION OF THE INVENTION

### MODIFIED RELEASE MILNACIPRAN FORMULATIONS

The milnacipran composition incorporates two types of modified-release dosage forms, namely delayed release and extended release.

Delayed-release portion is designed to prevent drug release in the upper part of the gastrointestinal (GI) tract. Delayed release can be achieved using enteric coatings. The enteric coated formulation remains intact or substantially intact in the stomach but dissolves and releases the contents of the dosage form once it reaches the small intestine. The purpose of an enteric coating is to delay the release of milnacipran within the stomach, thereby avoiding nausea, vomiting, or bleeding due to irritation of the gastric mucosa, which would otherwise result.

The delay in the release of milnacipran postpones the rise of milnacipran in the blood plasma for up to 4 hours after oral administration, hence allowing for bed time (PM) administration. The milnacipran blood plasma level for once-a-day formulation is the lowest 24 hours after the dose is taken. Since the intensity of centrally mediated side effects is controlled by drug blood plasma level, it is expected that the intensity of side effects would also be the lowest 24 hours after the last dose is taken. Milnacipran patients taking immediate release formulation twice-a-day and suffering from insomnia would be able to significantly decrease this side effect associated with milnacipran treatment by switching to PM administration. A once-a-day formulation when taken at bed time provides up to about a four-hour window during which essentially no drug is released, allowing a patient to fall a sleep and most likely enter the rapid eye movement (REM) sleep. Since milnacipran induces only minor disturbances of REM sleep compared with SSRIs and tricyclic antidepressants (Gervasoni D. et al., 2002, Pharmacol. Biochem. Behav., 73:557-563), minimal sleep disturbances are expected when the formulation is administered at bed time. Thus a once-a-day modified release milnacipran formulation provides the versatility of AM or PM dosing.

The milnacipran extended-release portion extends and maintains drug release within the intestines over a period of time before returning to the steady-state level at night time to avoid sleep disturbances. As used herein, “about” means approximately plus or minus ten percent.

The expected therapeutic benefit of these formulations is further supported by the results of a 12-week randomized, double-blind placebo-controlled dose escalation monotherapy trial that evaluated milnacipran in patients with a diagnosis of Fibromyalgia Syndrome (FMS) presented by Cypress Bioscience, Inc. at the 41^{st} Annual Meeting of American College of Neuropsychopharmacology, San Juan, Puerto Rico (Gendreau R. M. et al., Dec. 9, 2002, Poster presentation, Poster# 85 “Development of milnacipran, a dual reuptake inhibitor for treatment of chronic pain associated with fibromyalgia”).

In the FMS trial conducted by Cypress Bioscience, all patients were escalated over a 4-week period in weekly steps from 25 mg daily, to 50, 100, and finally 200 mg daily, or until dose-limiting toxicity was evident. The current available immediate release (IR) milnacipran formulation was used as the only milnacipran dosage form in this study. Patients who successfully reached the 200 mg daily dose were then treated for an additional 8 weeks at that dose. It is important to emphasize that at any given dose level, milnacipran once daily (QD-IR) patients received the full dose of immediate release milnacipran in the morning and received a placebo at night. Milnacipran twice daily (BID-IR) patients received the same total amount in a split dose, given morning and evening.

The primary endpoint used by Cypress Bioscience was defined as the change in pain score from baseline to endpoint based on pain scores collected on the patient electronic diary. Endpoint was defined as week twelve for assessments with a single value (such as clinical measures) or the average of scores at weeks 11 and 12 for diary-based outcomes. It was shown that milnacipran effectively treated pain associated with fibromyalgia syndrome and, additionally, improved mood in depressed patients with FMS. The improvement in pain scores reported by study participants, when 200 mg daily dose was reached, indicates that this substantially higher dose than the one typically used for depression treatment is needed to the alleviation of pain. On a 1-7 scale the global pain scores for all patients who reached endpoint at the time of the analysis, where 1 is very much improved, 4 is unchanged, and 7 is very much worse, the mean value for milnacipran patients was 2.3, while the mean value for placebo patients was 4.3 (the difference between the milnacipran groups and placebo is statistically significant at p=0.0001). Importantly, within the milnacipran groups, twice daily dosing was significantly more effective than once daily dosing in pain reduction. Twice daily dosing regimen in addition to being more therapeutically effective, also demonstrated fewer dose-related adverse events and resulted in a lower rate of dose intolerance than once daily regimen (19% of participants in QD-IR group failed the dose escalation vs. only 6% in BID-IR group). Note that no dose escalation failures were recorded in the placebo group.

These clinical differences between QD-IR and BID-IR are most likely due to the distinct differences in the drug plasma levels (especially Cₘₐₓ) that these two dosing regiments support. The BID-IR dosing regimen supports drug plasma levels characterized by lower Cₘₐₓ and lower drug plasma fluctuations over 24 hour time period than that of QD-IR. When a daily dose is administered QD-IR, the Cₘₐₓ is approximately twice higher than that of BID-IR dosing regimen. Higher Cₘₐₓ causes an increase in the severity of the adverse side effects (that also might interfere with the objective pain level self-assessment by the patient) and leads to a lower drug tolerance and patient compliance. Therefore, the observed superior milnacipran performance when drug was administered BID-IR is thought to be due to more “sustained” drug plasma levels over a 24 hour period.

Based on the clinical trial data obtained and presented by Cypress Bioscience, sleep quality improves, albeit marginally, when milnacipran was administered BID-IR. This could be interpreted as another indication that the formulation that provides more “sustained” drug plasma levels over a 24 hour period should demonstrate superior performance when compared to standard immediate release formulation and, importantly, cause less insomnia.

### Definitions

Delayed release dosage form: A delayed release dosage form is one that releases a drug (or drugs) at a time other than promptly after administration.

Extended release dosage form: An extended release dosage form is one that allows at least a twofold reduction in dosing frequency as compared to that drug presented as a conventional dosage form (e.g. as a solution or prompt drug-releasing, conventional solid dosage form).

Modified release dosage form: A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are the types of modified release dosage forms.

### Milnacipran

Milnacipran and methods for its synthesis are described in U.S. Pat. No. 4,478,836. Milnacipran (midalcipran, midacipran, F 2207) inhibits the uptake of both, norepinephrine (NE) and serotonin (5-HT), with an NE to 5-HT ratio of 2:1 (Moret et al., 1985, Neuropharmacology, 24:1211-1219; Palmier et al., 1989, Eur. J. Clin. Pharmacol., 37:235-238) but does not affect the uptake of dopamine. Milnacipran has no affinity for alpha or beta adrenergic, muscarinic, histaminergic, and dopaminergic receptors. This suggests that milnacipran has a low potential to produce anticholinergic, sedative, and stimulant effects. Milnacipran does not affect the number of beta adrenoceptors in rat cortex after chronic administration (Briley M. et al., Int. Clin. Psychopharmac., 1996, 11: 10-14). Additional information regarding milnacipran may be found in the Merck Index, 12^{th} Edition, at entry 6281.

As used herein “milnacipran” also encompasses pharmaceutically acceptable, pharmacologically active derivatives of milnacipran including both individual enantiomers of milnacipran (dextrogyral and levrogyral enantiomers) and their pharmaceutically acceptable salts, mixtures of milnacipran enantiomers and their pharmaceutically acceptable salts, and active metabolites of milnacipran and their pharmaceutically acceptable salts, unless otherwise noted. It is understood that in some cases dosages of enantiomers, derivatives, and metabolites may need to be adjusted based on relative activity of the racemic mixture of milnacipran.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization of diastereomeric salts/derivatives or preparative chiral chromatography.

### Combinations with Other Active Compounds

The milnacipran can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

Specific examples of compounds that can be adjunctively administered with milnacipran include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

By adjunctive administration is meant simultaneous administration of the compounds, in the same dosage form, simultaneous administration in separate dosage forms, and separate administration of the compounds.

### Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et.al., (Media, PA: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose,including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The amount of active agent released in each dose will be a therapeutically effective amount. In the case of milnacipran, the total amount in the dosage form is in the range of approximately 25 to 500 mg.

### Extended Release Dosage Rorms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray- congealed or congealed and screened and processed.

### Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Kit Containing Delayed Release/extended Release Formulations

A kit is provided wherein the once a day modified release dosage form is packaged to provide a method to conveniently begin dose titration at lower doses, for example, beginning at 25mg, gradually increasing to 50 mg, 75 mg, 100 mg, 200 mg, 400 mg, 500 mg, over a period ranging from three days up to 16 weeks. The kit wherein the packaging material may be a box, bottle, blister package, tray, or card. The kit will include a package insert instructing the patient to take a specific dose at a specific time, for example, a first dose on day one, a second higher dose on day two, a third higher dose on day three, and so on, until a maintenance dose is reached.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

All publications cited are incorporated by reference.

### Administration of Milnacipran Formulations

The formulation can be administered to any patient in need thereof. Although preferred patients are human, typically any mammal including domestic animals such as dogs, cats and horses, may also be treated.

The amount of the active ingredients to be administered is chosen based on the amount which provides the desired dose to the patient in need of such treatment to alleviate symptoms or treat a condition.

Milnacipran has been used as an antidepressant in approximately 400,000 patients, and is known to be non-toxic in humans. Pharmacokinetic studies have shown that oral doses of milnacipran are rapidly absorbed and extensively distributed in the body within 1-2 hours. Maximum plasma levels are quickly reached, with a half-life in humans of approximately 8 hours. Metabolism in the liver leads to the formation of ten chemically identified metabolites, although these metabolites represent only about 10% of the concentration of the parent drug. In humans, 90% of the parent drug is eliminated unchanged via the kidneys. This pharmacokinetic profile gives milnacipran certain pharmacokinetic advantages, such as low inter-individual variation in plasma levels, low potential for drug interactions, and limited impact on hepatic cytochrome P-450 systems. These pharmacokinetic properties differentiate milnacipran from most other antidepressant drugs and contribute to the good safety profile of milnacipran (Puozzo C. et al., 1996, Int. Clin. Psychopharmacol., 11:15-27; Caccia S., 1998, Clin. Pharmacokinet., 34:281-302; Puozzo C. et al., 1998, Eur. J. Drug Metab. Pharmacokinet., 23:280-286).

Milnacipran can be administered for the treatment of depression, for fibromyalgia syndrome, chronic fatigue syndrome, pain, attention deficit/hyperactivity disorder, and visceral pain syndromes (VPS) such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache.

Adverse reactions to the oral administration of milnacipran typically include at least one of the following: nausea, vomiting, headache, dyspepsia, abdominal pain, insomnia, tremulousness, anxiety, panic attack, palpitations, urinary retention, orthostatic hypotension, diaphoresis, chest pain, rash, weight gain, back pain, constipation, vertigo, increased sweating, agitation, hot flushes, tremors, fatigue, somnolence, dysoria, nervousness, dry mouth, and irritability.

The vomiting reflex is triggered by stimulation of chemoreceptors in the upper GI tract and mechanoreceptors in the wall of the GI tract which are activated by both contraction and distension of the gut wall as well as by physical damage. A coordinating center in the central nervous system controls the emetic response. The center is located in the parvicellular reticular formation in the lateral medullary region of the brain. Afferent nerves to the vomiting center arise from the abdominal splanchic and vagal nerves, vestibule-labyrinthine receptors, the cerebral cortex and the cehmoreceptors trigger zone (CTZ). The CTZ lies adjacent in the area postrema and contains chemoreceptors that sample both blood and cerebro spinal fluid. Direct links exist between the emetic center and the CTZ. The CTZ is exposed to emetic stimuli of endogenous origin and to stimuli of exogenous origin such as drugs. The efferent branches of the cranial nerves V, VII, and IX, as well as the vagus nerve and sympathetic trunk produce the complex coordinated set of muscular contractions, cardiovascular responses and reverse peristalsis that characterizes vomiting. The area postrema is rich in dopamine receptors as well as 5-hydroxytryptamine (5HT) receptors.

When administered orally, the extended release formulation first passes through the stomach, releasing 0-10% of the total milnacipran dose and then enters the intestines where drug is released slowly. The release profile is typically characterized by a 0.05-4 hours lag time period during which about 0-10% of the total milnacipran dose is released followed by a slow or extended drug release. The pharmaceutical composition of milnacipran provides the in vivo drug plasma levels characterized by Tₘₐₓ at 4-10 hours, preferably at 5-8 hours and an approximately linear drop-off sometime thereafter and Cₘₐₓ below 3000 ng/ml, preferably below 2000 ng/ml, and most preferably below 1000 ng/ml. This dosage form offers many advantages when compared to immediate release delivery systems, such as minimization of peak-trough-fluctuations, avoidance of undesirable side effects and/or lowering their intensity/severity, reduced frequency of administration and improved patient compliance.

This formulation is designed to be administered once-a-day to a patient in need thereof, so that milnacipran is delivered over approximately 24 hours, with diminished incidence and decreased intensity of one or more common milnacipran side effects such as nausea, vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

### EXEMPLIFICATION

The present invention will be further understood by reference to the following non-limiting examples.

### Example 1

### Preparation of a Delayed Release/Extended Release Milnacipran Tablet Using an Aqueous Granulation.

Ingredients, manufacturing process, and in vitro dissolution data for the extended release portion of the delayed release/extended release milnacipran pharmaceutical composition (Lot# 1, small scale manual batch):

| INGREDIENTS | mg per tablet |
|---|---|
| Milnacipran HCl | 120 |
| Hydroxypropyl | 150 |
| Methylcellulose E10M | |
| Ethyl cellulose 10cps | 70 |
| Dibasic Calcium | 100 |
| phosphate, Dihydrate | |
| Povidone K 90 | 8 |
| Magnesium stearate | 6 |
| Total tablet weight | 454 |

A wet granulation process consisting of dry blending, wet granulation, drying, size reduction, and final blending with lubricant steps, was utilized at the bench scale. The tablets were compressed using a single station bench top model tablet press.

Dissolution in Phosphate Buffer pH 6.8

| | | | | | | | | | | |
|---|---|---|---|---|---|---|---|---|---|---|
| Dissolution | 0.5 | 1 | 2 | 4 | 6 | 8 | 10 | 12 | 14 | 16 |
| time, hours | | | | | | | | | | |
| Milnacipran | 18.7 | 26.6 | 37.9 | 52.9 | 63.2 | 70.6 | 75.9 | 79.6 | 82.4 | 84.5 |
| released, % of | | | | | | | | | | |
| total dose | | | | | | | | | | |

USP dissolution apparatus I (rotating baskets at 100 rpm) filled with phosphate buffer, pH 6.8 was used for dissolution experiments. Experiments were carried out at 37° C. The analysis of dissolution samples was performed using UV method.

### Example 2

### Preparation of Alternative Delayed Release/Extended Release Milnacipran Tablet Using an Alcohol Granulation.

Ingredients, manufacturing process, and in vitro dissolution data for the extended release portion of the delayed release/extended release milnacipran pharmaceutical composition (Lot# 2, small scale manual batch).

| INGREDIENTS | mg per tablet |
|---|---|
| Milnacipran HCl | 200 |
| Lactose | 150 |
| Hydroxypropyl | 150 |
| methylcellulose | |
| K15M | |
| Povidone K 90 | 10 |
| Magnesium stearate | 5 |
| Total tablet weight | 515 |

A wet granulation process consisting of dry blending, wet granulation, drying, size reduction, and final blending with lubricant steps, was utilized at the bench scale. The tablets were compressed using a single station bench top model tablet press.

Dissolution in DI water

| | | | | | | | | | |
|---|---|---|---|---|---|---|---|---|---|
| Dissolution time, hours | 0.5 | 1 | 2 | 4 | 6 | 8 | 10 | 12 | 14 |
| Milnacipran released, | 14 | 22 | 33 | 48 | 59 | 67 | 72 | 76 | 85 |
| % of total dose | | | | | | | | | |

USP dissolution apparatus I (rotating baskets at 100 rpm) filled with DI water was used for dissolution experiments. Experiments were carried out at 37° C. The analysis of dissolution samples was performed using UV method.

### Example 3

### Preparation of a Delayed Release/Extended Release Milnacipran Tablet Using an Aqueous Granulation.

Ingredients, manufacturing process, and in vitro dissolution data for the extended release portion of the delayed release/extended release milnacipran pharmaceutical composition (bench—small scale manual batch, lab-equip—lab-equipment scale granulator or blender was used in batch preparation):

| | Lot# 3 - | Lot# 4 - | Lot# 5 - | Lot# 6 - |
|---|---|---|---|---|
| INGREDIENTS | bench | lab-equip | lab-equip | bench |
| Milnacipran HCl | 120 mg | 120 mg | 120 mg | 120 mg |
| Hydroxypropyl | 80 mg | | 150 mg | 150 mg |
| Methylcellulose K100M | | | | |
| Hydroxypropyl | 80 mg | 150 mg | | |
| Methylcellulose E10M | | | | |
| Dibasic Calcium | 150 mg | 118 mg | 98 mg | |
| phosphate, Dihydrate | | | | |
| Emcocel 50M | | | | |
| Lactose Anhydrous | | | | 98 mg |
| Ethocel 10cps | | 52 mg | 52 mg | 52 mg |
| Povidone K 90 | 8 mg | 8 mg | | |
| Aquacoat 30D | | | 3.7 mg | 5.7 mg |
| Magnesium stearate | 6 mg | 6 mg | 6 mg | 6 mg |
| Total tablet weight | 444 mg | 454 mg | 429.7 mg | 431.7 mg |

A wer granulation process consisting of dry blending, wet granulation, drying, size reduction, and final blending with lubricant steps, was utilized at the bench scale. The tablets were compressed using a single station bench top model tablet press.

Dissolution in Phosphate Buffer pH 6.8

| | |
|---|---|
| Dissolution | Milnacipran released, % of total dose |

| time, min | Lot# 3 | Lot# 4 | Lot# 5 | Lot# 6 |
|---|---|---|---|---|
| 30 | 21.2 | 19.9 | 18.0 | 18.4 |
| 60 | 30.1 | 29.2 | 26.0 | 26.6 |
| 120 | 42.5 | 42.2 | 37.5 | 38.2 |
| 180 | 51.6 | 51.6 | 46.4 | 47.0 |
| 240 | 58.9 | 59.0 | 53.7 | 54.2 |
| 300 | 64.9 | 64.9 | 59.7 | 60.3 |
| 360 | 70.0 | 69.8 | 65.1 | 65.5 |
| 480 | 77.9 | 77.0 | 73.0 | 73.6 |
| 600 | 83.4 | 82.0 | 78.4 | 79.5 |
| 720 | 87.3 | 85.4 | 82.9 | 83.7 |
| 840 | 90.1 | 88.1 | 85.9 | 86.9 |
| 960 | 92.3 | 90.2 | 88.2 | 88.6 |

USP dissolution apparatus I (rotating baskets at 100 rpm) filled with phosphate buffer, pH 6.8 was used for dissolution experiments. Experiments were carried out at 37° C. The analysis of dissolution samples was performed using UV method.

### Example 4

### Preparation of Alternative Delayed Release/Extended Release Milnacipran Tablet Using an Aqueous Granulation.

Ingredients, manufacturing process, and in vitro dissolution data for the extended release portion of the delayed release/extended release milnacipran pharmaceutical composition (small scale manual batch Lot# 7 and pilot batch Lot# 8, both aqueous granulation)

| | Lot# 7 - manual batch | Lot# 8 - pilot scale |
|---|---|---|
| Ingredients | mg per tablet | mg per tablet |
| Milnacipran HCl | 120 | 120 |
| Hydroxypropyl | 150 | 150 |
| Methylcellulose K100M | | |
| Emcocel 50M | 98 | 98 |
| Ethocel 10cps | 52 | 52 |
| Aquacoat 30D | 6 | 6 |
| Magnesium stearate | 6 | 6 |
| Total tablet weight | 432 | 432 |

A wet granulation process consisting of dry blending, wet granulation, drying, size reduction, and final blending with lubricant steps, was utilized at the bench scale. The tablets were compressed using a single station bench top model tablet press. The pilot batch was prepared using Zanchetta RotoP10 (high shear granulator) for aqueous wet granulation process. The drying was performed in Glatt GPCG-5 Fluid bed Granulator and the final blending was done using a “V” blender. The obtained blend was compressed using a rotary tablet press.

Dissolution in Phosphate Buffer pH 6.8

| | Milnacipran released, % of total dose | |
|---|---|---|
| Incubation time, min | Lot# 7 - manual batch | Lot# 8 - pilot scale |
| 30 | 15.5 | 16.2 |
| 60 | 23.2 | 24.6 |
| 120 | 34.5 | 36.7 |
| 180 | 43.7 | 46.4 |
| 240 | 51.7 | 54.6 |
| 300 | 58.2 | 61.5 |
| 360 | 63.7 | 67.3 |
| 480 | 72.1 | 76.5 |
| 600 | 78.4 | 83.6 |
| 720 | 83.1 | 88.8 |
| 840 | 86.5 | 92.3 |
| 960 | 88.9 | 94.7 |

USP dissolution apparatus I (rotating baskets at 100 rpm) filled with phosphate buffer pH 6.8 was used for dissolution experiments. Experiments were carried out at 37° C. The analysis of dissolution samples was performed using UV method.

### Example 5

### Preparation of Alternative Delayed Release/Extended Release Milnacipran Using an Aqueous Granulation.

Ingredients, manufacturing process, and in vitro dissolution data for the delayed release/extended release milnacipran pharmaceutical composition. EUDRAGIT L 100-55 (trade name ACRYL-EZE) was used to create delayed release coating around extended release cores. Lot# 8 extended release core tablets (see Example 4) were coated in a 24″ Accelacota Pan and the samples with the various delayed release coating content (weight gain, w/w) were collected. The samples were subjected to the in vitro dissolution tests that mimic the in vivo conditions to which tablet is exposed when administered orally (approximately 2 hours in the stomach at acidic pH followed by approximately 16-18 hours in the intestines at neutral pH (Multiparticulate Oral Drug Delivery, 1994, Ghebre-Sellassie I., Ed., Marcel Dekker, Inc.; Wilding I. R., 2001, Adv. Drug Deliv. Rev., 46:103-124).

In Vitro dissolution data for delayed release/extended release tablets. USP dissopution apparatus I (rotating baskets at 100 rpm) was used. The diddolution media was 0.1 N HCI for first 2 hours followed by phosphate buffer, pH 6.8. All dissolution tests were conducted at 37° C. UV method was used for the sample alysis. Total drug released (%) is given as a function of the incubation time.

| Cumulative | | | | | |
|---|---|---|---|---|---|
| Incubation time, | Lot# 9 | Lot# 10 | Lot# 11 | Lot# 12 | Lot# 13 |
| min (beginning | 6.36% | 8.39% | 10.29% | 11.01% | 12.74% |
| with 0.1 N HCl, | (weight | (weight | (weight | (weight | (weight |
| changing to | gain) DR | gain) DR | gain) DR | gain) DR | gain) DR |
| pH 6.8 buffer) | coating | coating | coating | coating | coating |
| 0.1 N HCl | | | | | |
| 30 | 0 | 0 | 0 | 0 | 0 |
| 60 | 0 | 0.11 | 0 | 0 | 0 |
| 120 | 2.52 | 0.94 | 0 | 0 | 0 |
| pH 6.8 buffer | | | | | |
| 150 | 20.07 | 18.78 | 17.92 | 20.24 | 21.32 |
| 180 | 29.13 | 28.28 | 28.29 | 31.42 | 33.31 |
| 240 | 41.25 | 40.97 | 41.89 | 45.70 | 47.27 |
| 300 | 50.06 | 50.61 | 51.91 | 56.12 | 57.33 |
| 360 | 57.18 | 58.58 | 60.14 | 64.31 | 65.33 |
| 420 | 63.20 | 65.21 | 67.10 | 71.19 | 71.87 |
| 480 | 68.38 | 70.82 | 72.92 | 77.00 | 76.69 |
| 600 | 76.69 | 79.8 | 82.31 | 86.39 | 82.21 |
| 720 | 83.09 | 86.73 | 88.95 | 93.11 | 89.53 |
| 840 | 87.81 | 91.62 | 93.80 | 97.97 | 94.85 |
| 960 | 91.11 | 95.06 | 97.39 | 101.48 | 98.64 |
| 1080 | 93.95 | 97.89 | 99.67 | 104.38 | 104.39 |

### Example 6

### An Alternative Extended Release Core Tablet

An extended release core tablet was prepared as described above. Preferred values and ranges are provided.

Extended Release Core Tablet (Lot# 14 - 2,000 tablets pilot batch)

| | | | Preferred |
|---|---|---|---|
| | | | range, % |
| | mg per core | % per core | per core |
| Ingredient | tablet | tablet | tablet |
| Milnacipran HCI | 120 | 27.8 | 10-80 |
| HPMC K100 M premium | 150 | 34.7 | 10-45 |
| Avicel pH 102 | 98 | 22.7 | 5-35 |
| Ethocel 10 cps | 52 | 12.0 | 0-40 |
| Aqua coat ECD 30 | 6 | 1.4 | 0-10 |
| Magnesium stearate | 6 | 1.4 | 0.25-5 |
| Total extended release core tablet | 432 | | |
| weight | | | |

### Example 6

### An Alternative Delayed Release Coated Tablet

Lot# 14 extended release core tablet was used to prepare a pilot batch of enteric coated tablets. Delayed release/extended release tablets Lot# 15 (2,000 tablets pilot batch for bioavailability study) were prepared as described above however, an additional Opadry seal coat was applied on the extended release core prior to delayed release coat application. Preferred values and ranges are provided below.

Delayed release coat Lot# 15 (2,000 tablets pilot batch
for bioavailability study)

| | | | Preferred |
|---|---|---|---|
| | | | range, % |
| | | % weight | weight gain |
| | mg per core | gain per | per core |
| Ingredient | tablet | core tablet | tablet |
| Opadry ® 7006 clear (Colorcon) | 8.6 | 2 | 0-10 |
| ACRYL-EZE | 34.6 | 8 | 4-20 |

### Example 7

### Pharmacokinetics of Delayed Release/Extended Release Formulation

Delayed release/extended release tablet Lot# 15 was used in a bioavalability study (see Examples 5 and 6 for formulation ingredients and manufacturing procedure).

In vitro dissolution data for Lot# 15 delayed release/extended release tablets is given below. USP dissolution apparatus I (rotating baskets at 100 rpm) was used. The dissolution media was 0.1 N HCI for first 2 hours followed by phosphate buffer, pH 6.8. All dissolution tests were conducted at 37° C. The following HPLC method was used for the sample analysis: column Inertsil ODS-3V, 4.6×250 mm; detection wavelength 230 nm, injection volume 20 microL, mobile phase Buffer: Methanol (40:60) mixture. Buffer was prepared by addition of 1 ml of TEA to 400 ml of 50 mM sodium dihydrogen orthophosphate solution. pH was adjusted to 3 with orthophosphoric acid.

| Cumulative Dissolution | |
|---|---|
| time, hours | |
| (beginning with 0.1 N HCl, | Lot# 15 |
| changing to pH 6.8 buffer) | Milnacipran released, % of total dose |
| 0.1 N HCl | |
| 2 | 0.28 |
| pH 6.8 buffer | |
| 2.5 | 10.05 |
| 3 | 18.34 |
| 4 | 30.74 |
| 5 | 41.40 |
| 6 | 49.70 |
| 7 | 56.56 |
| 8 | 61.49 |
| 10 | 72.94 |
| 12 | 79.68 |
| 14 | 86.15 |
| 16 | 89.48 |
| 18 | 93.72 |

The bioavailability study to determine the concentration-time plasma profile was done on male healthy subjects with the mean age 24 years (range: 20 to 35 years). The study was conducted as a single-dose study.

Milnacipran 120 mg delayed release/extended release tablets corresponding to the formulation of Example 6 (Lot# 15) were administered to the 12 healthy subjects. Prior to tablet administration subjects were given standard breakfast.

Blood samples were collected prior to dosing (0 hour) and at 0.5, 1.0, 1.5, 2.0, 2.5, 3.0, 3.5, 4.0, 4.5, 5.0, 5.5, 6.0, 7.0, 8.0, 9.0, 10.0, 12.0, 14.0, 16.0, 18.0, 20.0, and 24.0 hours after the dose. Plasma samples were assayed for milnacipran using a validated high performance liquid chromatographic procedure (LC/MS).

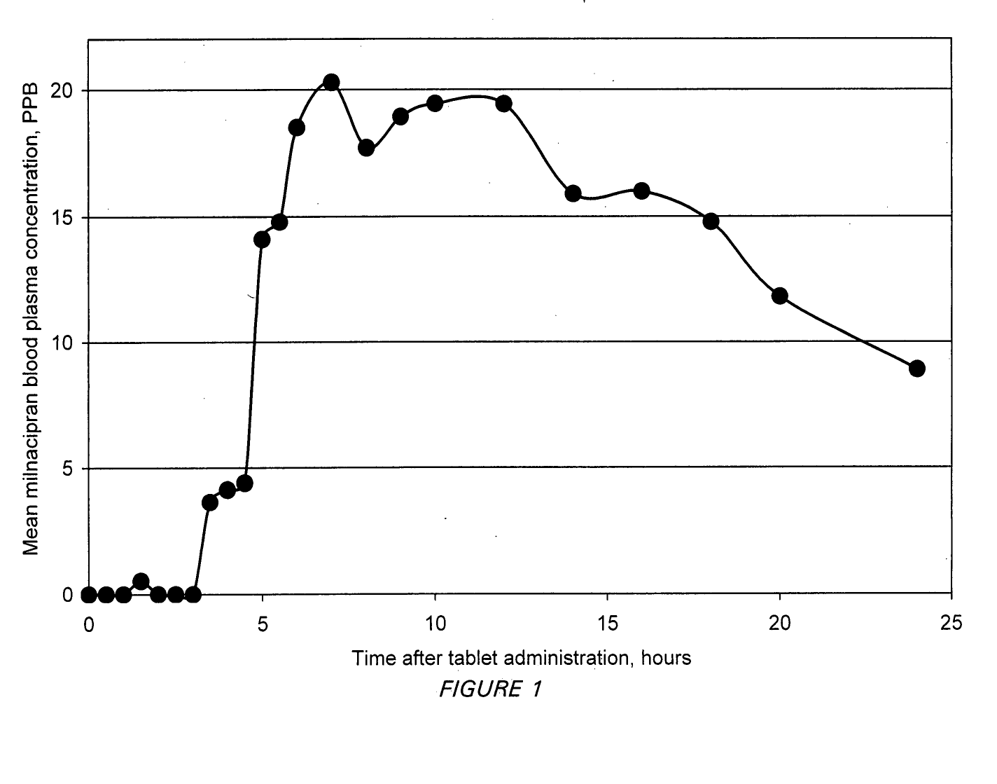

The mean plasma concentration-time profile for Milnacipran 120 mg delayed release/extended release tablets is given in FIG. 1.

We claim:

1. A milnacipran formulation that provides delayed or extended release of milnacipran to produce a therapeutic effect over approximately 24 hours when administered to a patient in need, with diminished incidence and reduced intensity relative to one or more immediate release milnacipran side effects.

2. The milnacipran formulation according to claim 1, wherein the side effect is nausea.

3. The malnacipran formulation according to claim 1, wherein the side effects are selected from the group consisting of vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

4. The milnacipran formulation according to claim 1 having a milnacipran release profile that is characterized by release of less than approximately 10% of the total dose over a period up to four hours, followed by a slow or extended drug release.

5. The milnacipran formulation according to claim 4 wherein the defined period of time is between approximately four and approximately twenty-four hours.

6. The milnacipran formulation according to claim 1 providing milnacipran blood plasma levels that are characterized by Tₘₐₓ at 4-10 hours, and Cₘₐₓ below approximately 3000 ng/ml.

7. The milnacipran formulation according to claim 6 providing milnacipran blood plasma levels that are characterized by Cₘₐₓ below approximately 2000 ng/ml.

8. The milnacipran formulation according to claim 6 providing milnacipran blood plasma levels that are characterized by Cₘₐₓ below approximately 1000 ng/ml.

9. The milnacipran formulation according to claim 1 further comprising at least one other active compound selected from the group consisting of analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

10. The milnacipran formulation according to claim 9 comprising compounds selected from the group consisting of aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil, molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

11. The milnacipran formulation according to claim 1, wherein the milnacipran is in the form of a therapeutically equivalent dose of dextrogyral or levrogyral enantiomers of the milnacipran or pharmaceutically acceptable salts thereof.

12. The milnacipran formulation according to claim 1, wherein the milnacipran is in the form of a therapeutically equivalent dose of a mixture of milnacipran enantiomers or pharmaceutically acceptable salts thereof.

13. The milnacipran formulation according to claim 1, wherein the milnacipran is in the form of a therapeutically equivalent dose of the active metabolite of milnacipran or pharmaceutically acceptable salts thereof.

14. The milnacipran formulation according to claim 1, wherein the milnacipran is in the form of a therapeutically equivalent dose of para-hydroxy-milnacipran (F2782) or pharmaceutically acceptable salts thereof.

15. The milnacipran formulation according to claim 1 comprising an enteric coating.

16. The milnacipran formulation according to claim 1, wherein the administrable milnacipran unit dose is from 25 to 500 mg.

17. The milnacipran formulation according to claim 1, wherein the administrable milnacipran unit dose is from 200 to 500 mg.

18. The formulation according to claim 9 comprising 25 to 500 mg milnacipran and 100 to 600 mg modafinil.

19. A milnacipran formulation that allows extended release of a theraupetically effective amount of milnacipran over approximately 24 hours when administered to a patient in need, comprising
an extended-release milnacipran formulation coated with an enteric coating, wherein the enteric coated formulation remains intact or substantially intact in the stomach but dissolves and releases the contents of the dosage form once it reaches the small intestine, over a period of time resulting in therapeutic milnacipran blood plasma levels for an extended period of time before returning to the steady-state level at night time to avoid sleep disturbances.

20. A kit comprising the milnacipran formulation of claim 1.

21. The kit of claim 20 comprising different dosage units of milnacipran to allow for dosage escalation.

22. The kit of claim 20 comprising instruction on taking the formulation once daily before bedtime.

23. A method of making a milnacipran formulation comprising providing the formulation of claim 1.

24. A method for delivering a therapeutic dose of milnacipran to a patient in need thereof, with diminished incidence or reduced intensity of common milnacipran side effects, comprising administering to the patient in need thereof the milnacipran formulation of claim 1.
